(19) United States
(12) Patent Application Publication
(10) Pub. No.: US 2012/0088839 A1
(43) Pub. Date: Apr. 12, 2012

(54) COMPOSITION FOR REGULATING CELLULAR SENESCENCE COMPRISING [N-2-(CYCLOHEXYLOXYL)-4-NITROPHENYL]-METHANESULFONAMIDE

(76) Inventors: Sang Chul PARK, Seongnam-shi (KR); Jeong A. HAN, Chuncheon-si (KR)

(21) Appl. No.: 13/306,711

(22) Filed: Nov. 29, 2011

Related U.S. Application Data
(62) Division of application No. 12/600,447, filed on Nov. 16, 2009.

(30) Foreign Application Priority Data
May 15, 2007 (KR) 10-2007-0047015

(51) Int. Cl. A61K 31/18; A61P 25/16; A61P 25/14; A61P 25/00; A61P 19/02; C12N 5/071; A61P 5/00; A61P 17/14; A61P 19/10; A61P 9/10; A61P 7/02; A61P 27/02; A61P 25/28; A61P 17/00

(52) U.S. Cl. 514/605; 435/375

(57) ABSTRACT

The present invention relates to a composition for inhibiting cellular senescence, comprising N-[2-(cyclohexyloxyl)-4-nitrophenyl]-methanesulfonamide.

### CROSS-REFERENCE PARAGRAPH

The present application is a divisional of U.S. patent application Ser. No. 12/600,447, filed Nov. 16, 2009 (pending), which is a national phase of PCT Application No. PCT/KR2008/002688, filed May 14, 2008, which claims the benefit of priority to Korean application number 10-2007-0047015, filed May 15, 2007, the contents of which are incorporated by reference herein in their entirety.

### TECHNICAL FIELD

The present invention relates to a composition for inhibiting cellular senescence, comprising N-[2-(cyclohexyloxyl)-4-nitrophenyl]-methanesulfonamide.

### BACKGROUND ART

Cellular senescence plays an important role in complex biological processes, including development, aging, and tumorigenesis, and many attempts have been made to understand some of its fundamental features. However, it is still unclear what the mechanism of aging is. Meanwhile, the hypothesis that reactive oxygen species produced in the process of aerobic metabolism damage cells and are main causes of aging is persuasive (1). Particularly, in connection with this, the molecular inflammation hypothesis of aging was recently proposed that a transcriptional factor NF-κB activated by ROS induces the expression of pro-inflammatory genes, such as cyclooxygenase-2 (COX-2) and iNOS, is induced, and reactive oxygen species and reactive nitrogen species are produced by these genes, and thus cell damage is accelerated, leading to the progression of senescence (2).

This is supported by reports that NF-κB activity was increased in the heart, liver, kidneys and brain of aged mice or rats (3), and that the expression of the NF-κB gene in human keratinocytes led to cellular senescence (4). In addition, there are reports that the expression of pro-inflammatory genes, such as COX-2, iNOS, IL-1β and TNF-α among the target genes of NF-κB was increased in the brain, kidneys and spleen of aged mice (5-9). Moreover, DNA microarray studies showed that the expression of pro-inflammatory genes, such as COX-2, IL-1β, MCP-1, Gro-α and ICAM-1, increase in senescent human aged skin fibroblasts (10 and 11).

COX-2 is a key molecule in the molecular inflammation hypothesis. This is an enzyme that produces prostaglandin H2 (PGH2) from arachidonic acid and oxygen, in which PGH2 is a precursor for prostaglandin synthesis. Among two isoforms of COX, COX-1 is expressed at a constant level, whereas the expression of COX-2 is induced by various stimuli to synthesize many, various types of prostaglandins (12). Among the final products of COX, prostaglandin E2 (PGE2) is an important substance causing inflammatory reactions, and most of nonsteroidal anti-inflammatory drugs developed to date inhibit the enzymatic active site of COX. Among the nonsteroidal anti-inflammatory drugs, aspirin, ibuprofen, flurbiprofen and indomethacin, which have been frequently used, inhibit the enzymatic activities of COX-1 and COX-2 in a non-selective manner. In recent years, inhibitors capable of selectively inhibiting COX-1 and COX-2 have been developed, and it is known that the selective COX-2 inhibitors have a very potent anti-inflammatory activity, even though the selective COX-1 inhibitors also have an anti-inflammatory activity (13).

If A molecular inflammation is a critical factor for aging, the COX-2 inhibitors must be able to delay the senescence processes. There are several reports about the effects of non-specific COX inhibitors on aging processes, and according to the papers, cognitive decline resulting from senescence was delayed in women to which ibuprofen was administered for a long term (14). However, the long-term administration of salicylic acid, acetylsalicylic acid or indomethacin to Drosophila led to a decrease in the average lifespan of the Drosophila or had no effect on the average lifespan (15). As for the cellular senescence, aspirin inhibited senescence in human vascular endothelial cells, whereas indomethacin promoted senescence, in which case the inhibitors were regulated senescence by regulating the production of nitrogen monoxide and reactive oxygen species, but not by inhibiting the enzymatic activity of COX (16).

As described above, although various theories for senescence have recently been proposed, it is yet unclear whether the pro-inflammatory activity of COX-2 is involved in the aging process and whether the COX-2 inhibitors can prevent senescence. It is expected that the establishment of such a senescence mechanism will be important in reversing senescence and treating senescence-associated diseases requiring the recovery of normal physiological functions, for example, Werner syndrome, Hutchinson-Gilford syndrome, etc.

### DISCLOSURE OF THE INVENTION

Accordingly, the present inventors have examined and investigated inhibiting activity of selective COX-2 inhibitors in a cellular senescence model of human skin fibroblasts for study and, as a result, have found that among the COX-2 inhibitors, particularly N-[2-(cyclohexyloxyl)-4-nitrophenyl]-methanesulfonamide, regulates cellular senescence regardless of the enzymatic activity of COX-2 and is closely connected with the regulation of expression of caveolin-1, thereby completing the present invention.

### TECHNICAL SOLUTION

It is an object of the present invention to provide a composition for inhibiting cell senescence, comprising N-[2-(cyclohexyloxyl)-4-nitrophenyl]-methanesulfonamide.

Other objects and advantages of the present invention will be apparent from the following detailed description, the appended claims and the accompanying drawings.

### ADVANTAGEOUS EFFECTS

As described above, the present invention relates to a composition for inhibiting cellular senescence, comprising N-[2-(cyclohexyloxyl)-4-nitrophenyl]-methanesulfonamide.

Among three selective COX-2 inhibitors used in the experiments of the present invention, only NS-398, which is N-[2-(cyclohexyloxyl)-4-nitrophenyl]-methanesulfonamide, inhibited cellular senescence, the remaining celecoxib and nimesulide promoted cellular senescence. In addition, all of three non-selective COX inhibitors (aspirin, ibuprofen and flurbiprofen) all promoted cellular senescence.

During the progression of cellular senescence, the expression of COX-2 was decreased, whereas the enzymatic activity of COX-2 was increased, and the cellular senescence regulatory activity of the three selective COX-2 inhibitors have no connection with the concentration of reactive oxygen species in cells, the activity of NF-κB and the amounts of p53 and p21 proteins. However, it was found that the three selective COX-2 inhibitors regulated the expression of caveolin-1 at the transcriptional level and regulated the intracellular total cholesterol level, and that these results were closely connected with the cellular senescence regulatory activity of the three selective COX-2 inhibitors.

In addition, it was found that the three selective COX-2 inhibitors stimulated collagen synthesis in cells and suppressed the activities of the matrix metalloproteinases MMP-2 and MMP-9.

The above results suggest that the enzymatic activity of COX-2 does not mediate the process of cellular senescence, N-[2-(cyclohexyloxyl)-4-nitrophenyl]-methanesulfonamide of the present invention inhibits cellular senescence through the mechanism associated with the regulation of expression of caveolin-1, but not through the inhibition of COX-2 enzyme activity, and the composition comprising the compound can regulate individual senescence.

### BRIEF DESCRIPTION OF THE DRAWINGS

FIG. 1A is a graphic diagram showing the results of measurement of the number of population doublings (PD) for cells, treated with DMSO (vehicle control group) or the selective COX-2 inhibitor NS-398 (20 μM), celecoxib (1 μM) and nimesulide (20 μM), respectively, in which the cells had a number of population doublings of 24 before the treatment; FIG. 1B is a photograph of the cells of FIG. 1A, taken after the cells were seeded on a 35-mm dish and subjected to SA-β-gal staining; FIG. 1C is a graphic diagram showing the ratio of SA-β-gal (+) cells in a total of 100 cells randomly counted under an optical microscope; FIG. 1D is a graphic diagram showing the results of measurement of population doublings (PD) for cells, treated with the nonselective COX inhibitors aspirin (1 mM), ibuprofen (20 μM) and flurbiprofen (5 μM) and control DMSO, respectively; FIG. 1E is a SA-β-gal staining photograph of the cells of FIG. 1D; and 1F is a graphic diagram showing the ratio of SA-β-gal (+) cells in the cells of FIG. 1E. Herein, the error bars in FIGS. 1C and 1F indicate the mean standard deviation of two independent experiments performed in duplicate. *P<0.05 (Mann-Whitney U-test, compared to the DMSO-treated cells).

FIG. 2A shows the results of Western blot analysis of COX-1 and COX-2, conducted after collecting the fibroblasts of a donor (1) and a donor (2) in each passage and extracting the total protein from the collected cells. In FIG. 2A, β-actin was used as a loading control. FIG. 2B is a graphic diagram showing the results of measurement of the concentration of prostaglandin E2 in each cell culture at each passage and shows that prostaglandin E2 increases in the senescence process (*P<0.05 (Mann-Whitney U-test, compared to P15 cells), and FIGS. 2C and 2D are graphic diagrams showing the results of measurement of the concentrations of prostaglandin E2 after treatment with selective COX-2 inhibitors (FIG. 2C) and nonselective COX inhibitors (FIG. 2D), respectively, and show that the COX-2 inhibitors effectively inhibit the production of prostaglandin E2. In FIG. 2, the concentration of prostaglandin E2 was analyzed in collected cell cultures and corrected with the number of cells. Also, the error bars indicate the mean standard deviation of two independent experiments performed in triplicate. *P<0.05 (Mann-Whitney U-test, compared to DMSO-treated cells).

FIG. 3A is a graphic diagram showing fluorescence analysis results for cell extracts, obtained by adding DCFH-DA to cells at each passage and culturing the cells at 37° C., and shows that the amount of reactive oxygen species increases in the senescence process. In FIG. 3A, the error bars indicate the mean standard deviation of three independent experiments performed in duplicate. *P<0.05 (Mann-Whitney U-test, compared to P15 cells). FIG. 3B is a graphic diagram showing the results of measurement of the change in the amount of reactive oxygen species in P15 and P29 cells, treated with selective COX-2 inhibitors, and shows that the amount of reactive oxygen species did not change in the P15 cells, but changed in the P29 cells. In FIG. 3B, the error bars indicate the mean standard deviation of three independent experiments performed in duplicate. *P<0.05 (Mann-Whitney U-test, compared to DMSO-treated cells). FIGS. 3C and 3D show the results of Western blot analysis for the expression of the antioxidant enzymes catalase SOD-2 and Gpx-1 in the senescence process. Specifically, FIG. 3C shows the results of Western blot analysis for cells at each passage, and FIG. 3D shows the results of Western blot analysis for P28 cells cultured in the presence of selective COX-2 inhibitors.

FIG. 4 shows the results of measurement of the effects of COX-2 inhibitors on NF-κB activity in the cell senescence process. Specifically, FIG. 4A shows the results of Western blot analysis, conducted using the NF-κB p65 in cytosol fractions and nucleus fractions, extracted from cells at each passage (upper panel), and is a graphic diagram showing the results of densitometric measurement of the ratio of nucleus p65 relative to cytosol p65 (lower panel), and FIG. 4B shows the results of Western blot analysis for the amount of NF-κB p65 cytosol fractions and nucleus fractions, extracted from cells, which were cultured in the presence of inhibitors and harvested at P18 (upper panel), and is a graphic diagram showing the results of densitometric measurement of the ratio of nucleus p65 relative to cytosol p65 (lower panel).

FIG. 5 shows the results of Western blot analysis for the effects of selective COX-2 inhibitors on the expressions of p53 and p21. Specifically, FIG. 5 shows the results of measurement of the amounts of p53 (FIG. 5A) and p21 (FIG. 5B), extracted from cells, which were cultured in the presence of COX-2 inhibitors and harvested at each passage.

FIG. 6 shows the results of analysis for the effects of selective COX-2 inhibitors on the expression of caveolin-1 in the cell senescence process. Specifically, FIG. 6A shows the results of measurement of the amount of caveolin-1, extracted from cells, which were cultured in the presence of COX-2 inhibitors and harvested at each passage, FIG. 6B shows the amount of caveolin-1 in cells, treated with inhibitors at varying time points, FIG. 6C shows the expression levels of caveolin-1 in cells, treated either with NS-398 and DMSO (solvent for MG-132) or with NS-398 and 50 μM MG-132 (proteasome inhibitor), and FIG. 6D shows the results of RT-PCR, conducted using primers specific for caveolin-1 and GAPDH genes, after treating cells with inhibitors at each time point and extracting total RNA from the treated cells. In FIG. 6D, the GAPDH gene was used as a control group to determine the amount of total RNA in the cells in each condition (upper panel), and the amount of caveolin-1 mRNA (lower panel) was corrected with the amount of GAPDH. FIG. 6E is a graphic diagram showing the results of measurement of the concentration of total cholesterol in fat components, extracted from cells, which were cultured in the presence of inhibitors and harvested at each passage. In FIG. 6E, the cholesterol concentration was corrected with the protein concentration, and the error bars indicate the mean±standard deviation of two independent experiments performed in triplicate. *P<0.05 (Mann-Whitney U-test, compared to DMSO-treated cells).

FIG. 7A is a graphic diagram showing the synthesis of collagen in cells, which were treated with selective COX-2 inhibitors for 5 days. In FIG. 7A, the collagen values were corrected with the number of cells, and the error bars indicate the mean±standard deviation of three independent experiments performed in duplicate (*P<0.05 (Mann-Whitney U-test, compared to DMSO-treated cells)). FIG. 7B shows the results of zymographic analysis for the activities of matrix metallopeptidase-2 ((MMP-2; 67 kDa) and matrix metallopeptidase-9 (MMP-9; 84 kDa) in cell cultures, treated with inhibitors for 10 days. The results in FIG. 7B suggest that selective COX-2 inhibitors reduces the degradation of collagen by inhibiting the activities of matrix metallopeptidase-2 and matrix metallopeptidase-9 in fibroblasts.

### BEST MODE FOR CARRYING OUT THE INVENTION

The present invention relates to a composition for inhibiting cellular senescence, comprising N-[2-(cyclohexyloxyl)-4-nitrophenyl]-methanesulfonamide.

Hereinafter, the present invention will be described in further detail.

The present invention relates to a composition for inhibiting cellular senescence, comprising N-[2-(cyclohexyloxyl)-4-nitrophenyl]-methanesulfonamide. As used herein, the term “N-[2-(cyclohexyloxyl)-4-nitrophenyl]-methanesulfonamide” is a selective COX-2 inhibitor, which is a sulfonanilide represented by the following formula 1:

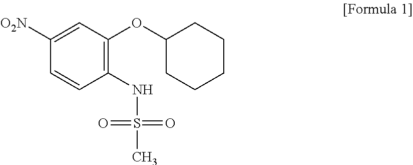

As used herein, the term “inhibiting cellular senescence” refers to a method of inhibiting senescence by inhibiting the synthesis of intracellular caveolin or of inducing senescence by inducing the synthesis of caveolin. Herein, caveolin includes all proteins and mRNA of caveolin-1, caveolin-2 and caveolin-3. In addition, the term “inhibiting cellular senescence” may include inhibiting cellular senescence through the intracellular metabolic pathway of collagen.

According to a preferred embodiment of the present invention, the inhibition of senescence in the present invention can regulate cellular senescence regardless of the intracellular reactive oxygen species pathway, the pathway of the transcriptional factor NF-κB, which sensitively responds to oxidative stress, and the intracellular p53 and p21 pathways, and can inhibit cellular senescence by, for example, inhibiting the synthesis of caveolin-1 through the caveolin-1 pathway. In addition, the inhibitor of the present invention can increase collagen synthesis and inhibit senescence by inhibiting the activities of matrix metallopeptidases (MMP-2 and MMP-9).

As used herein, the term “cells” means animal cells, preferably mammalian cells, more preferably human cells, and most preferably human fibroblast cells.

Although the composition of the present invention may be prepared as a composition for research purposes, it may also be prepared as a pharmaceutical composition. If the composition of the present invention is prepared as a pharmaceutical composition, it comprises, in addition to siRNA, a pharmaceutically acceptable carrier. In the pharmaceutical composition of the present, the pharmaceutically acceptable carrier may be a conventional one for formulation, including lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methylhydroxy benzoate, propylhydroxy benzoate, talc, stearic acid, magnesium and mineral oil, but is not limited thereto. The pharmaceutical composition according to the present invention may further comprise, in addition to these components, a lubricant, a wetting agent, a sweetener, a flavoring agent, an emulsifier, a suspending agent, a preservative, etc.

According to methods known to those skilled in the art, the pharmaceutical composition of the present invention can be formulated in unit dosage forms or multiple dosage formsusing a pharmaceutically acceptable carrier and/or vehicle. Herein, the formulation may be in the form of a solution, suspension or emulsion in oily or aqueous medium or in the form of an extract, powder, granule, tablet or capsule, and may additionally comprise a dispersant or a stabilizer. Suitable pharmaceutically acceptable carriers and formulations are described in Remington's Pharmaceutical Sciences (19^{th} ed., 1995).

The pharmaceutical composition of the present invention may be administered orally or parenterally. For parenteral administration, the composition can be administered by intravenous injection, subcutaneous injection, intramuscular injection, intraperitoneal injection or transdermal delivery. A preferred mode of administration is intravenous injection, which is systemic administration, subcutaneous injection, intramuscular injection, intraperitoneal injection or transdermal delivery.

The correct dosage of the pharmaceutical composition of the present invention will vary depending various factors, such as the particular formulation, the mode of application, age, body weight, sex and disease severity of the patient, diet, the time of administration, the route of administration, excretion rate and reaction sensitivities. It is understood that the ordinary skilled physician will readily be able to determine and prescribe a correct dosage of the pharmaceutical composition.

In another aspect, the present invention provides a method for inhibiting cellular senescence, which comprises treating aged cells with an effective amount of N-[2-(cyclohexyloxyl)-4-nitrophenyl]-methanesulfonamide.

In still another aspect, the present invention provides a method for regulating cellular senescence in a patent in need of regulation of cellular senescence, the method comprising administering an effective amount of N-[2-(cyclohexyloxyl)-4-nitrophenyl]-methanesulfonamide to the patient.

Although the methods of the present invention may be applied to any cells, cells, which are more important for therapeutic purposes, include: (a) cells with replicative capacity in the central nervous system, including astrocytes, endothelial cells, and fibroblasts which play a role in such age-related diseases as Alzheimer's disease, Parkinson's disease, Huntington's disease, and stroke, (b) cells with finite replicative capacity in the integument, including fibroblasts, sebaceous gland cells, melanocytes, keratinocytes, Langerhan's cells, and hair follicle cells which may play a role in age-related diseases of the integument, such as dermal atrophy, elastolysis and skin wrinkling, sebaceous gland hyperplasia, senile lentigo, graying of hair and hair loss, chronic skin ulcers, and age-related impairment of wound healing, (c) cells with finite replicative capacity in the articular cartilage, such as chondrocytes and lacunal and synovial fibroblasts which play a role in degenerative joint disease, (d) cells with finite replicative capacity in the bone, such as osteoblasts, bone marrow stromal fibroblasts, and osteoprogenitor cells which play a role in osteoporosis, (e) cells with finite replicative capacity in the immune system such as B and T lymphocytes, monocytes, neutrophils, eosinophils, basophils, NK cells and their respective progenitors, which may play a role in age-related immune system impairment, (f) cells with a finite replicative capacity in the vascular system including endothelial cells, smooth muscle cells, and adventitial fibroblasts which may play a role in age-related diseases of the vascular system including atherosclerosis, calcification, thrombosis, and aneurysms, and (g) cells with a finite replicative capacity in the eye such as pigmented epithelium and vascular endothelial cells which may play an important role in age-related macular degeneration.

According to a preferred embodiment of the present invention, cells suitable for the present invention are derived from mammalian cells such as human cells. More preferably, the cells in the present invention are fibroblasts.

Hereinafter, the present invention will be described in further detail with reference to examples. It is to be understood, however, that these examples are illustrative only, and the scope of the present invention is not limited thereto.

### I. EXAMPLES

In the experiments of the present invention, NS-398 (Cayman Chemical Co.) was used as N-[2-(cyclohexyloxyl)-4-nitrophenyl]-methanesulfonamide.

### Example 1

### Cell Culture

According to the literature of Boyce and Ham (1983), human fibroblasts were isolated from foreskin, and then cultured in a DMEM medium, containing 10% fetal bovine serum (Life Technology Inc., Grand Island, N.Y.), penicillin (100 units/ml) and streptomycin (100 units/ml)). General cultured cells showed a decrease in growth rate with an increase in passage number, and cells with a passage number higher than 30 showed completely arrested growth and started to show characteristic phenomena, such as replicative senescence reported in the prior art (Yeo et al., 2000 a and b).

### Example 2

### Experiment of Regulation of Growth Rate by COX-2 Inhibitors

In order to examine the effects of COX-2 inhibitors on cellular senescence, cells were treated with each of the three selective COX-2 inhibitors NS-398, celecoxib and nimesulide, the three nonselective COX-inhibitors aspirin, ibuprofen and flurbiprofen, inhibiting the activities of both COX-1 and COX-2, and DMSO (vehicle control group), and then the treated cells were stained using a general cell staining method in the following manner and were measured for population doublings (PDs). First, cells having a number of population doublings (PDs) of 24 were treated with each of DMSO (vehicle control group), NS-398 (20 μM), celecoxib (1 μM), nimesulide (20 μM), aspirin (1 mM), ibuprofen (20 μM) and flurbiprofen (5 μM), and were cultured. Then, the number of the cells was calculated by trypan blue staining, and the number of population doublings (PDs) of the cells was calculated according to the following equation 1:

Number of population doublings (PDs)=log(A/B)/log 2 [Equation 1]

wherein A is the number of cells harvested at one passage, and B is the initial cell number at that passage.

### Example 3

### Senescence-Associated Beta-Galactosidase (SA-β-gal) Staining

In order to examine the effects of COX-2 inhibitors on cellular senescence, cells were treated with each of the three selective COX-2 inhibitors, NS-398, celecoxib and nimesulide, the three nonselective COX inhibitors aspirin, ibuprofen and flurbiprofen, inhibiting the activities of both COX-1 and COX-2, and DMSO (vehicle control group), and then were subjected to senescence-associated β-galactosidase (SA-β-gal) staining. Herein, the senescence-associated β-galactosidase (SA-β-gal) staining was performed in the following manner according to the method of Dimri et al. (1995) (17). First, cells were treated with each of DMSO (vehicle control group), NS-398 (20 μM), celecoxib (1 μM), nimesulide (20 μM), aspirin (1 mM), ibuprofen (20 μM) and flurbiprofen (5 μM), and were cultured. The cultured cells were seeded on a 35 mm dish, and then stabilized. Then, the cells were washed twice with PBS and fixed with 3% formaldehyde at room temperature for 5 minutes. Then, the cells were treated once with PBS and stained with SA-β-gal solution (1 mg/ml X-gal, 40 mM citric acid/sodium phosphate, pH 6.0, 5 mM potassium ferrocyanide, 5 mM potassium ferricyanide, 150 mM sodium chloride, 2 mM magnesium chloride) at 37° C. for 24 hours. During the progression of the reaction, light was blocked. The stained cells were observed with a phase contrast microscope (Olympus, CK40) to measure the color development. Then, among the cells, a total of 100 cells were randomly counted, and the percentage of SA-β-gal (+) cells in the 100 cells was calculated. The experiment was independently repeated twice, and the mean value and standard deviation of the measurements were calculated.

### Example 4

### Analysis of Prostaglandin E2(PGE2)

In order to examine whether COX-2 inhibitors also have an effect on the amount of prostaglandin E2 (PGE2), cells were treated with COX-2 inhibitors in the same manner as in Example 2, and the culture medium of the cultured cells was analyzed with ELISA (Cayman Chemicals, Ann Arbor, Mich.) to measure the amount of PEG2 secreted from the culture medium, and the measured value was corrected with the cell number.

### Example 5

### Measurement of Reactive Oxygen Species

In order to examine the effects of COX-2 inhibitors on cellular senescence, cells were cultured and treated in the same manner as in Example 2, and reactive oxygen in the cells was then measured. The cultured cells were treated with 5 μM DCFH-DA (Invitrogen, Carlsbad, Calif.), and the cells were incubated at 37° C. for 45 minutes and then washed with PBS. The washed cells were collected in 1 ml PBS, and then disrupted with ultrasonic waves. The fluorescence of the cells was measured with a fluorescence spectrophotometer (Molecular Devices, Sunnyvale, Calif.), and the measured fluorescence value was corrected with the cell number.

### Example 6

### Western Blot

In order to examine the effects of COX-2 inhibitors on cellular senescence, cells were cultured and treated in the same manner as in Example 2, and then subjected to Western blot. The cultured cells were washed and collected in PBS, and then were disrupted in RIPA buffer (150 mM NaCl, 100 mM Tris-HCl, 1% Tween-20, 1% sodium deoxycholate and 0.1% SDS), containing 0.5 mM EDTA, 1 mM PMSF, 10 μg/ml leupeptin, 10 μg/ml aprotinin and 10 μg/ml pepstatin. The disrupted cells were centrifuged, and the supernatant was collected. Proteins in the cell extract were isolated by SDS-PAGE and transferred to nitrocellulose membranes. Then, the proteins were allowed to react with each of p53, p21, COX-1, COX-2 and caveolin-1 antibodies, and the protein-antibody complexes on the nitrocellulose membranes were allowed to react with peroxidase-conjugated anti-mouse or anti-rabbit secondary antibody. Then, the corresponding bands were visualized by chemiluminescence (Amersham Bioscience, Boston, Mass.) using an ECL kit. Herein, the antibodies for p53, p21 and COX-1 were purchased from Oncogene Science (Cambridge, Mass.), Cell Signaling Technology, Inc. (Danvers, Mass.), and Santa Cruz Biotechnology (Santa Cruz, Calif.), respectively, and the antibodies for COX-2 and caveolin-1 were purchased from BD Bioscience (San Jose, Calif.). Also, β-actin was used as an intracellular control protein to correct the amount of the intracellular total protein.

### Example 7

### RT-PCR

In order to examine the effects of COX-2 inhibitors on cellular senescence, cells were cultured and treated in the same manner as in Example 2, and then subjected to RT-PCR. The total RNA of the cells was extracted with TRIzol reagent (Life Technology Inc., Grand Island, N.Y.), and cDNA was synthesized from 0.5 μg of the total RNA using a reverse transcription (RT) kit (Qiagen, Valencia Calif.). The caveolin-1 gene was amplified using a sense primer (5′-ACA TCT CTA CAC CGT TCC CAT-3′) and an anti-sense primer (5′-TGT GTG TCC CTT CTG GTT CTG-3′), and the GAPDH gene was amplified using a sense primer (5′-TGT TGC CAT CAA TGA CCC CTT-3′) and an anti-sense primer (5′-CTC CAC GAC GTA CTC AGC G-3′). The polymerase chain reaction was performed in the following conditions: 25 cycles of 30 sec at 95° C., 30 sec at 60° C. and 30 sec at 72° C. The resulting DNA products were electrophoresed on 2% agarose gel containing EtBr.

### Example 8

### Analysis of Cholesterol

In order to examine the effects of COX-2 inhibitors on cellular senescence, cells were cultured and treated in the same manner as in Example 2, and then analyzed for cholesterol. 2×10⁶ cells were treated with a mixture of chloroform and methanol (2:1) to extract a fatty component. The amount of total cholesterol in the fatty component was measured at 570 nm using a staining method according to the manual of BioVision (Mountain View, Calif.), and the measured values were corrected with the protein concentration.

### Example 9

### Measurement of Collagen Biosynthesis

In order to examine the effects of COX-2 inhibitors on cellular senescence, cells were cultured and treated in the same manner as in Example 2, and then analyzed for collagen biosynthesis. The analysis of collagen biosynthesis was performed according to the method of Robert et al. (18). In brief, 5 μCi/ml of L-[2, 3-3H]-proline (Amersham Bioscience, Boston, Mass.) was added to the cells, which were then cultured for 24 hours. The culture medium and the cells were collected, and then the cells were disrupted with ultrasonic waves in 50 mM Tris-HCl (pH 7.2), containing 100 mM NaCl and 10 mM proline. Trichloroacetic acid (TCA) was added to each of the culture medium and the cell extract supernatant, and the precipitate was dissolved in 0.2 M NaOH and neutralized with 150 mM HCl and HEPES, and then bacterial collagenase was added to the solution. After the solution was centrifuged, the supernatants were combined, and then measured for radioactivity.

### Example 10

### Gelatin Gel Zymography

In order to examine the effects of COX-2 inhibitors on cellular senescence, cells were cultured and treated in the same manner as in Example 2, and then subjected to gelatin gel zymography. The cell culture medium was electrophoresed on SDS-PAGE gel containing 1 mg/ml gelatin. After the gel was washed with 2.5% Triton X-100, it was immersed in a solution, containing 50 mM Tris-HCl, 150 mM NaCl, 10 mM CaCl₂ and 0.02% NaN₂, and was then stained with 0.1% Coomassie blue solution.

### II. RESULTS OF EXAMPLES

### 1. COX-2 Inhibitors Regulate Cellular Senescence in Human Fibroblasts

In order to examine the effects of COX-2 inhibitors on cellular senescence, the cells were treated with each of the three selective COX-2 inhibitors (NS-398, celecoxib and nimesulide), the three nonselective COX inhibitors (aspirin, ibuprofen and flurbiprofen) inhibiting the activities of both COX-1 and COX-2, and DMSO (vehicle control group), and the number of population doublings in the cells was examined. Herein, the DMSO itself had no effect on the number of population doublings.

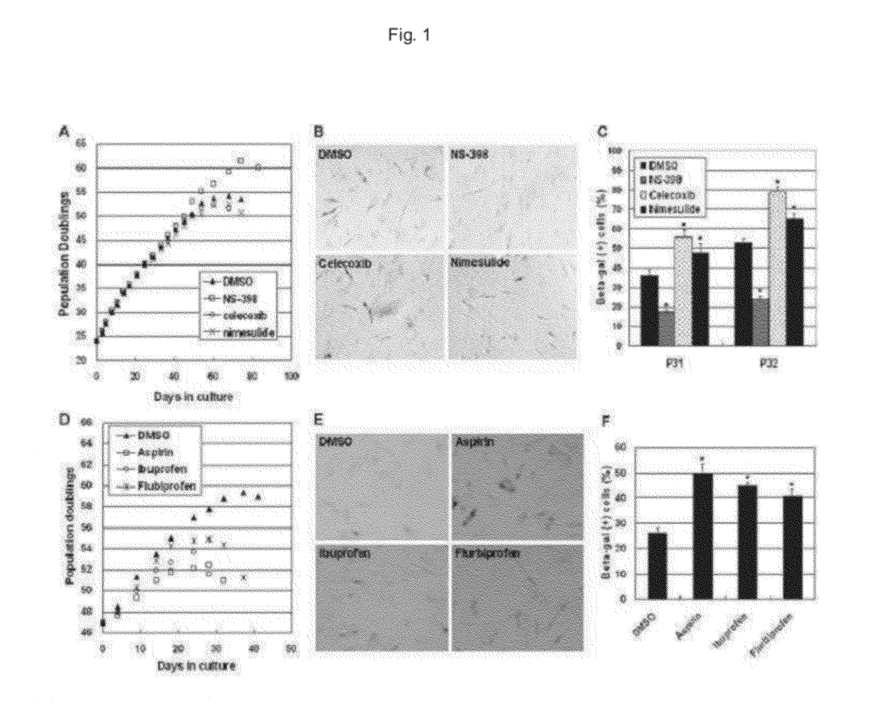

In the experiment, NS-398, which is one of the three selective COX-2 inhibitors, increased the maximum number of population doublings by 7 times compared to DMSO, whereas celecoxib and nimesulide reduced the maximum number of population doublings by two times (FIG. 1A). Also, with respect to the ratio of SA-3-gal positive cells as senescence markers, NS-398 reduced the ratio by two times compared to DMSO, whereas celecoxib and nimesulide increased the ratio by 1.5 times and 1.3 times, respectively (FIGS. 1B and 1C).

The nonselective COX inhibitors aspirin, ibuprofen and flurbiprofen reduced the maximum number of population doublings by 7 times, 5 times and 4 times, respectively, compared to DMSO (FIG. 1D), and increased the ratio of SA-3-gal positive cells by 1.9 times, 1.7 times and 1.6 times, respectively (FIGS. 1E and 1F).

Such results show that NS-398 strongly inhibits cellular senescence in human fibroblasts, whereas the other selective COX-2 inhibitors and the nonselective COX inhibitors promote cellular senescence.

### 2. COX-2 Inhibitors Regulate the Senescence of Human Fibroblasts Regardless of COX-2 Enzyme Activities

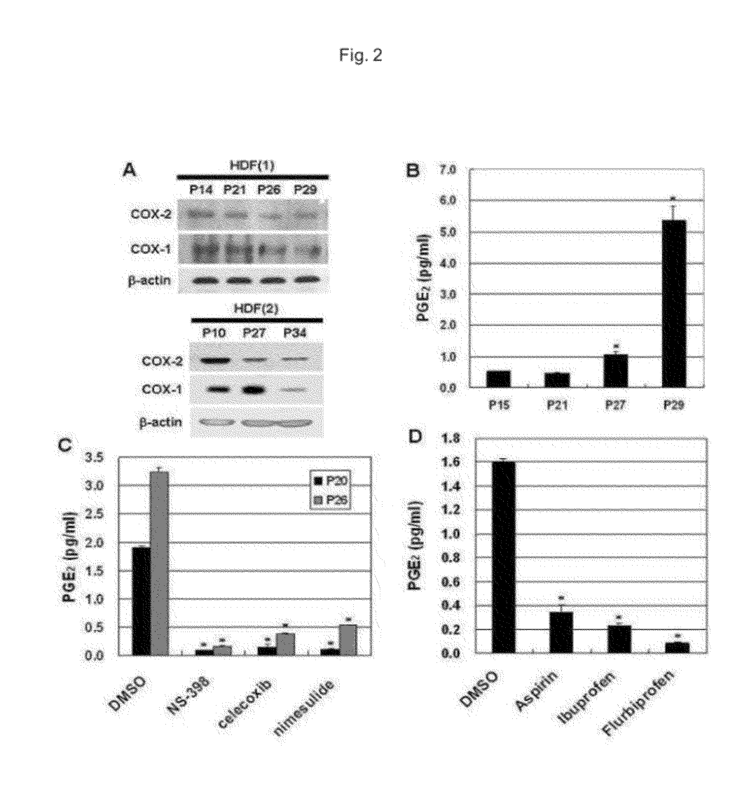

During the progression of cellular senescence, the expressions of COX-1 and COX-2 significantly decreased, and this reduction was observed in two kinds of different human fibroblasts (FIG. 2A). However, interestingly, the production of the final product prostaglandin E₂ significantly increased (FIG. 2B), suggesting that the enzyme activities increased, even though the expression of COX decreased.

When the cells were treated with the selective COX-2 inhibitors, the production of prostaglandin E₂ was almost completely blocked (FIG. 2C), indicating that the increase in prostaglandin E₂ caused by senescence is mainly attributable to the increase in COX-2 enzyme activity. Not only the selective COX-2 inhibitors, but also the nonselective COX inhibitors, almost completely inhibited the production of prostaglandin E₂ (FIG. 2D), indicating that the inhibitors used in this experiment effectively inhibited the enzymatic activity of COX-2 at each concentration.

The above results indicate that the COX-2 inhibitors regulate cellular senescence in human fibroblasts, and that this regulation is caused by a mechanism having no connection with the inhibition of COX-2 enzyme activity.

### 3. The Regulation of Cellular Senescence by Selective Cox-2 Inhibitors has No Connection with Reactive Oxygen Species

To establish the mechanism by which the selective COX-2 inhibitors regulate cellular senescence, the effects of the inhibitors on the generation of reactive oxygen species and the activity of NF-κB were examined.

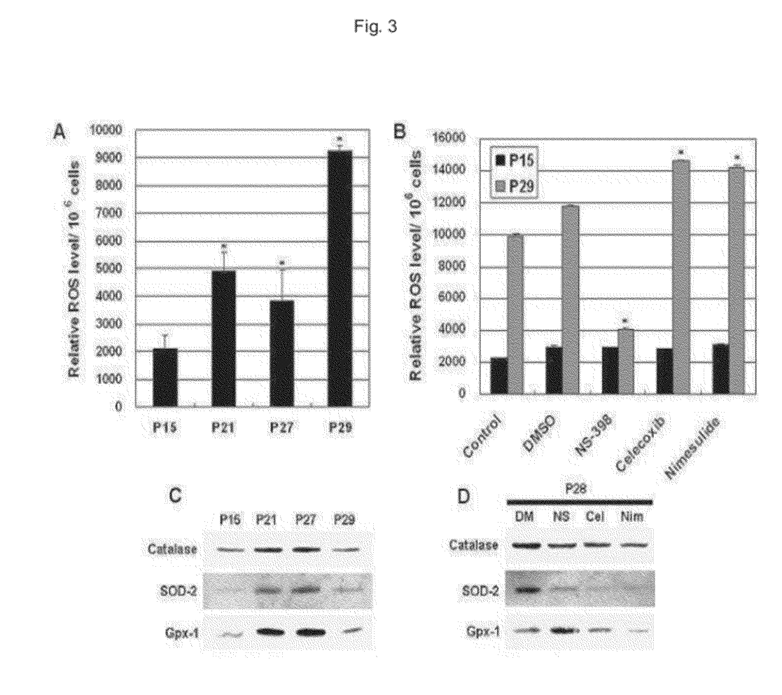

The level of intracellular reactive oxygen species was gradually increased during the cellular senescence process as reported in the prior art (FIG. 3A). When the cells were treated with the inhibitors for a long period of time (65 days), NS-398 reduced the level of reactive oxygen species by three times, whereas celecoxib and nimesulide increased the level by 1.2 times (FIG. 3B, P29). However, when the cells were treated with the inhibitors for a short period of time (7 days), the three drugs all had no effect on the level of reactive oxygen species (FIG. 3B, P15). This suggests that the selective COX-2 inhibitors have no effect on the generation of reactive oxygen species. The regulation of reactive oxygen species, which occurs when the cells are treated with the inhibitors for a long period of time, is thought to be a secondary phenomenon resulting from the cellular senescence regulatory effect of the inhibitors.

It was observed that the expressions of antioxidant enzymes, such as catalase, SOD-2 (superoxide dismutase-2) and Gpx-1 (glutathione peroxidase-1), were increased during the cellular senescence process (FIG. 3C). Specifically, NS-398 reduced the expressions of catalase and SOD-2 and increased the expression of Gpx-1. Celecoxib reduced the expressions of catalase and SOD-2 and had no effect on the expression of Gpx-1. Nimesulide reduced all the expressions of catalase, SOD-2 and Gpx-1 (FIG. 3D). As described above, the inhibitors had an effect on the expressions of the antioxidant enzymes, but this effect was not consistent with the senescence regulatory effect of the inhibitors. This suggests again that the selective COX-2 inhibitors do not regulate cellular senescence by regulating the generation of reactive oxygen species.

### 4. The Regulation of Cellular Senescence by Selective Cox-2 Inhibitors has No Correlation with the NF-κB Pathway

It was reported that the transcriptional factor

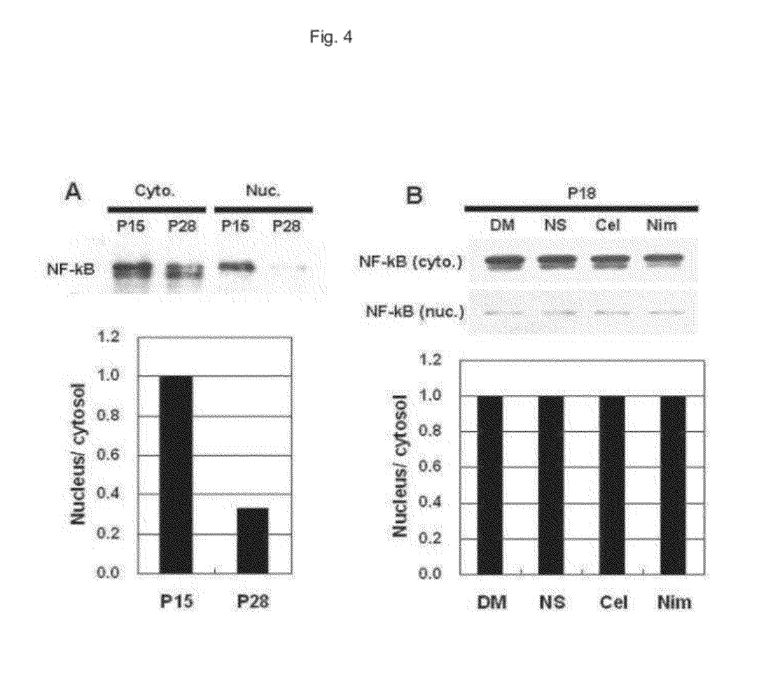

NF-κB sensitively responded to oxidative stress, and that the activity thereof was increased during the senescence process (19). However, in the case of human fibroblasts, the nuclear migration of NF-κB does remarkably reduced in aged cells compared to young cells (FIG. 4A), and the selective COX-2 inhibitors had no effect on the nuclear migration of NF-κB (FIG. 4B). Such results suggest that NF-κB dose not play a decisive role in the senescence process of human fibroblasts and that the cellular senescence regulatory effect of the selective COX-2 inhibitors has no correlation with the NF-κB pathway.

### 5. The Regulation of Cellular Senescence by the Selective COX-2 Inhibitors has No Correlation with the p53/p21 Pathway

It is well known that the p53/p21 pathway plays a key role in the senescence process of human fibroblasts. Accordingly, the effects of the selective COX-2 inhibitors on the expressions of p53 and p21 were examined.

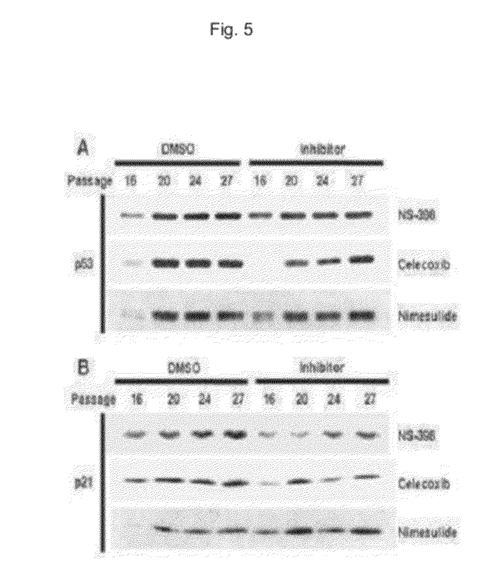

The expressions of p53 and p21 were increased during the cellular senescence process as reported in the prior art (FIGS. 5A and 5B). Specifically, NS-398 inhibited the expression of p21 without inhibiting the expression of p53. Celecoxib inhibited the expressions of both p53 and p21. Nimesulide increased the expressions of p53 and p21 (FIGS. 5A and 5B). Such results indicate that the inhibitors had an effect on the p53/p21 pathway. However, this effect was not consistent with the senescence regulatory effects of the inhibitors. This suggests that the selective COX-2 inhibitors do not regulate cellular senescence through the p53/p21 pathway.

### 6. The Senescence Regulatory Effects of the Selective COX-2 Inhibitors are Closely Connected with the Expression of Caveolin-1

Caveolin-1 is another molecule that is known to play a key role in the senescence process of human fibroblasts (20). To establish the mechanism by which the selective COX-2 inhibitors regulate cellular senescence, the effects of the inhibitors on the expression of caveolin-1 were examined.

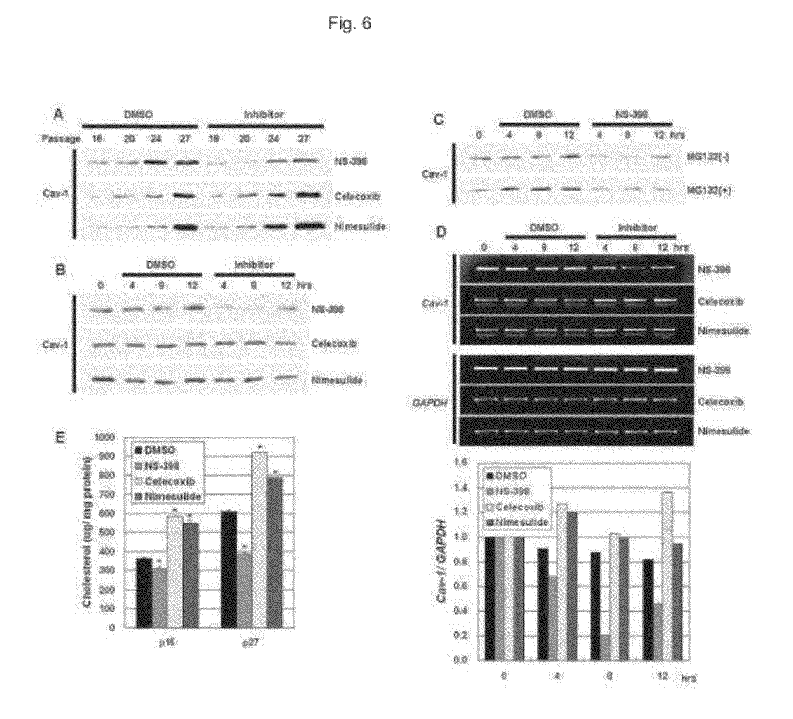

The expression of caveolin-1 was increased in the senescence process as reported in the prior art (FIG. 6A). Specifically, NS-398 inhibited the expression of caveolin-1 at all passages, whereas celecoxib and nimesulide increased the expression of caveolin-1 (FIG. 6A). NS-398 clearly inhibited the expression of caveolin-1, even when the cells were treated with NS-398 only for 4 hours. However, celecoxib and nimesulide did not significantly change the expression of caveolin-1, when the cells were treated with each of celecoxib and nimesulide for a short period of time (FIG. 6B). Such results were consistent with the cellular senescence regulatory effects of the selective COX-2 inhibitors, suggesting that the cellular senescence regulatory effects of the selective COX-2 inhibitors are closely connected with the expression of caveolin-1.

Because NS-398 had an excellent effect of inhibiting the expression of caveolin-1, an experiment was performed to examine whether NS-398 reduces the expression of caveolin-1 through protein degradation by proteasome. As shown in FIG. 6C, the inhibitory effect of NS-398 against the expression of caveolin-1 was not recovered by the proteasome inhibitor MG132. This indicates that NS-398 does not inhibit the expression of caveolin-1 through protein degradation by proteasome.

If so, the possibility for the expression of caveolin-1 to be regulated at the transcriptional level is very high. In order to confirm this possibility, the mRNA level of caveolin-1 was measured. In the experimental results, NS-398 reduced the mRNA level of caveolin-1, whereas celecoxib and nimesulide increased the level (FIG. 6D). This strongly suggests that the selective COX-2 inhibitors regulate cellular senescence by regulating the expression of caveolin-1 at the transcriptional level.

It is known that cholesterol is an important regulatory factor in the expression of caveolin-1 (21). Accordingly, the effects of the selective COX-2 inhibitors on the intracellular total cholesterol concentration were examined. The intracellular total cholesterol concentration was increased in aged cells by 1.7 times compared to in young cells (FIG. 6E), as reported in the prior art (22). Meanwhile, NS-398 reduced the total cholesterol concentration, whereas celecoxib and nimesulide increased the total cholesterol concentration (FIG. 6E). Such results suggest that the selective COX-2 inhibitors have a high possibility of regulating the expression of caveolin-1 through the regulation of the intracellular cholesterol concentration, thus regulating cellular senescence.

### 7. The Selective COX-2 Inhibitors Improve Collagen Metabolism in Skin Fibroblasts

It is known that intrinsic skin aging has a close connection with a decrease in the content of collagen in the dermal layer. Biochemically, the content of collagen is determined by the balance between the rate of collagen synthesis by dermal fibroblasts, and the rate of collagen degradation by matrix metalloproteinases secreted from fibroblasts and keratinocytes. As individual senescence progresses, the rate of collagen synthesis in skin fibroblasts is decreased, whereas the rate of collagen degradation by matrix metalloproteinases is increased, leading to a decrease in the content of collagen in the skin dermal layer (23).

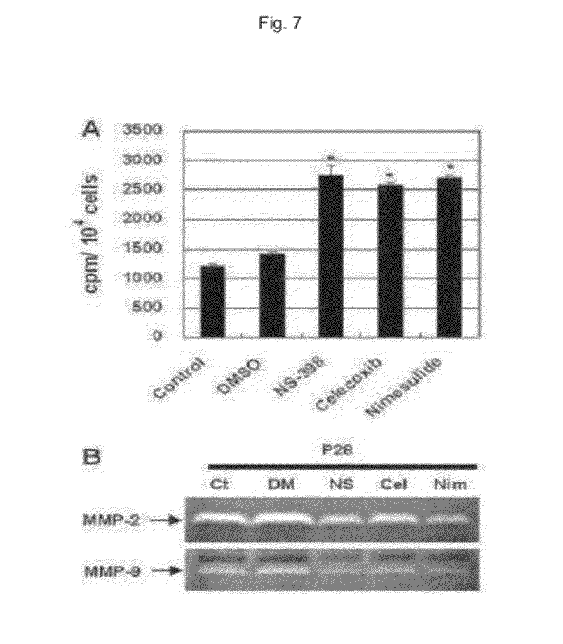

Because it was observed that the selective COX-2 inhibitors regulated the senescence of skin fibroblasts, the effects of the inhibitors on collagen metabolism were examined. Interestingly, the three selective COX-2 inhibitors all increased the rate of collagen synthesis in skin fibroblasts by about two times (FIG. 7A), and reduced the activities of matrix metalloproteinase-2 and matrix metalloproteinase-9 (FIG. 7B). Such results indicate that COX-2 enzyme activity is involved in collagen metabolism, and the three selective COX-2 inhibitors all can inhibit actual cellular senescence, even though the selective COX-2 inhibitors showed different effects on cellular senescence.

### III. CONSIDERATIONS

It was recently suggested that COX-2 mediated individual and cellular senescence through inflammatory enzyme activity (2 and 24). However, the function of COX-2 in the individual and cellular senescence processes is not yet clear. The present inventors have found that the two selective COX-2 inhibitors and the three nonselective COX inhibitors promote cellular senescence (FIG. 1), suggesting that the enzymatic activity of COX-2 does not mediate cellular senescence, at least in human fibroblasts. Also, the three selective COX-2 inhibitors showed different effects on cellular senescence (FIG. 1A), indicating that the cellular senescence regulatory effects of the COX-2 inhibitors are attributable to a mechanism having no connection with the enzymatic activity. Such results are consistent with the previous report that aspirin inhibits cellular senescence in human vascular endothelial cells, whereas indomethacin promotes cellular senescence, and this promotion is not attributable to the inhibition of COX enzyme activity, but is attributable to the regulation of production of nitrogen monoxide and reactive oxygen species (16).

It is known that not only the nonselective COX inhibitors, but also the selective COX-2 inhibitors, have various physiological activities having no connection with the inhibition of enzyme activity. For example, the selective COX-2 inhibitors, such as NS-398 and nimesulide, remove reactive oxygen species from human promonocytes (25). Also, NS-398 and celecoxib regulate the expressions of p21 and p27, but also the activities of NF-κB, ERK and Akt (26). However, in the present invention, the evidence that the three selective COX-2 inhibitors have effects on the generation of reactive oxygen species in fibroblasts or the activity of NF-κB was not found (FIGS. 3B and 4B). Also, although the inhibitors had an effect on the expressions of p53 and p21, this effect had no connection with the cellular senescence regulatory effect of the inhibitors (FIGS. 5A and 5B). Rather, the present inventors have found that the selective COX-2 inhibitors regulate the expression of caveolin-1, and this regulation has a close connection with the cellular senescence regulatory effect of the inhibitors (FIG. 6A).

Caveolae is a dented portion in the cell membrane and is known to play an important role in the endocytosis process. Caveolin is the major structural protein of caveolae and includes three isoforms: caveolin-1, caveolin-2 and caveolin-3. Among them, caveolin-1 is expressed in most cells and is known to interact with various signaling molecules, such as epithelial growth factor receptor, G protein and protein kinase C (27). Recently, it was reported that caveolin-1 is an important protein determining cellular senescence in human fibroblasts. The expression of caveolin-1 is increased in aged cells and attenuates growth signals by binding to epithelial growth factor receptor (20). Also, when the expression of caveolin-1 in aged cells is reduced, the synthesis of DNA is initiated again, and the shape of the cells is returned to a shape like that of the aged cells (28 and 29).

The present inventors have found that the selective COX-2 inhibitors regulate the expression of caveolin-1 and the concentration of cholesterol (FIGS. 6A and 6E), and this finding has important meanings in several terms below. First, this finding emphasizes again that receptor-mediated signaling is important to retain youthfulness at the cell level (probably, also at the individual level). This is because not only caveolin-1, but also cholesterol, has a strong effect on receptor-mediated signaling (30). Second, this finding indicates that caveolin-1 can be used as a new target of the selective COX-2 inhibitors, and thus the inhibitors can provide new molecular bases when they are developed into senescence regulatory drugs.

The transcriptional factor NF-κB is a key molecule in the molecular inflammation hypothesis of aging (2). When NF-κB is activated by reactive oxygen species, inflammatory genes such as COX-2 are expressed to cause senescence. However, the present inventors have observed that, in the case of human fibroblasts, the activity of NF-κB and the expression of COX-2 is reduced in the cellular senescence process, indicating that the molecular inflammation hypothesis is not correct, at least in human fibroblasts (FIGS. 2A and 4A). The previous reports that the activity of NF-κB did not change or rather decreased in the senescence process of human fibroblasts support the conclusions of the present inventors (3 and 31).

According to the present invention, the production of prostaglandin E2 was increased due to the activity of COX-2 in the senescence process of fibroblasts (FIGS. 2B and 2C). However, interestingly, the expression of the COX-2 protein was reduced in the senescence process (FIG. 2A), suggesting that the COX-2 enzyme activity itself was increased in the senescence process. With respect to the increase in the enzyme activity, two descriptions are possible. First, hydroperoxide, such as alkyl peroxide or peroxynitrite, is required in order for a cyclooxygenase reaction to occur (12). It was reported that the generation of reactive oxygen species, including alkyl peroxide and peroxynitrite, was increased in the cellular senescence and individual senescence processes (32). In the present invention, it was confirmed again that the generation of reactive oxygen species was increased in the cellular senescence process (FIG. 3A). Thus, as the generation of reactive oxygen species was increased in the senescence process, the enzymatic activity of COX-2 would possibly be increased. Second, it was reported that, in the case of human lung fibroblasts, the COX substrate arachidonic acid in a culture medium of aged cells was increased (33). Because free fatty acid rapidly reaches equilibrium inside and outside cells, the increase in arachidonic acid in the culture medium indicates that arachidonic acid in cytoplasm was also increased. Thus, because the concentration of the substrate arachidonic acid in cytoplasm was increased, the enzymatic activity of COX-2 would possibly be increased.

The decrease in collagen synthesis and the increase in matrix metalloproteinase activity are important causes of skin senescence (23), and the senescence of skin fibroblasts and keratinocytes provides a good description for this change in collagen metabolism during the skin senescence process. This is because, as cellular senescence progresses, the synthesis of collagen in fibroblasts is reduced (34), and the activities of matrix metalloproteinases in fibroblasts and keratinocytes are increased (24 and 35). The present inventors have found that the three selective COX-2 inhibitors all increase the synthesis of collagen in fibroblasts and inhibit the activities of matrix metalloproteinases (FIG. 7). This suggests that COX-2 enzyme activity is closely connected with collagen metabolism. It was also reported in the previous studies that prostaglandin E2 derived from COX-2 inhibited the expression of collagen in hepatic stellate cells, and NS-398 increased the expression of collagen in fibroblasts and hepatic stellate cells (24 and 36). In view of the importance of collagen metabolism in skin senescence, the possibility for the selective COX-2 inhibitors to inhibit skin senescence is high. Thus, it is valuable to test the effects of the COX-2 inhibitors as skin anti-senescence drugs.

The present inventors have found that the selective COX-2 inhibitors regulate senescence at the cell level according to a mechanism having no connection with enzyme activity. However, the exact function of COX-2 in the senescence process remains unclear. Accordingly, in the future, there is a need to find the function of COX-2 in the senescence process and to study the effects of the COX-2 inhibitors at the individual level.

### REFERENCES

1. Finkel, T., and Holbrook, N. J. (2000) Nature 408, 239-247
2. Chung, H. Y., Kim, H. J., Kim, K. W., Choi, J. S., and Yu, B. P. (2002) Microsc. Res. Tech. 59, 264-272
3. Helenius, M., H?nninen, M., Lehtinen, S. K., and Salminen, A. (1996) Biochem. J. 318, 603-608
4. Bernard, D., Gosselin, K., Monte, D., Vercamer, C., Bouali, F., Pourtier, A., Vandenbunder, B., and Abbadie, C. (2004) Cancer Res. 64, 472-481
5. Kim, H. J., Kim, K. W., Yu, B. P., and Chung, H. Y. (2000) Free Radic. Biol. Med. 28, 683-692
6. Manev, H., Uz, T., and Qu, T. (2000) Exp. Gerontol. 35, 1201-1209
7. Kim, J. W., Baek, B. S., Kim, Y. K., Herlihy, J. T., Ikeno, Y., Yu, B. P., and Chung, H. Y. (2001) J. Gerontol. A. Biol. Sci. Med. Sci. 56, B350-355
8. Yang, B., Larson, D. F., and Watson, R. R. (2004) Life Sci. 75, 655-657
9. Gelinas, D. S., and McLaurin, J. (2005) Neurochem. Res. 30, 1369-1375
10. Shelton, D. N., Chang, E., Whittier, P. S., Choi, D. H., and Funk, W. D. (1999) Curr. Biol. 9, 939-945
11. Yoon, I. K., Kim, H. K., Kim, Y. K., Song, I. H., Kim, W. K., Kim, S. Y., Baek, S. H., Kim, J. H., and Kim, J. R. (2004) Exp. Gerontol. 39, 1369-1378
12. Smith, W. L., Garavito, M., and DeWitt, D. L. (1996) J. Biol. Chem. 271, 33157-33160
13. Flower, R. J. (2003) Nat. Rev. Drug Discov. 2, 179-191
14. Kang, H. J., and Grodstein, F. (2003) Neurology 60, 1591-1597
15. Massie, H. R., Williams, T. R., and Iodice, A. A. (1985) J. Gerontol. 40, 257-260
16. Bode-B?ger, S. M., Martens-Lobenhoffer, J., T?ger, M., Schr?der, H., and Scalera, F. (2005) Biochem. Biophys. Res. Commun. 334, 1226-1232
17. Dimri, G. P., Lee, X., Basile, G., Acosta, M., Scott, G., Roskelley, C., Medrano, E. E., Linskens, M., Rubelj, I., Pereira-Smith, O., Peacocke, M., and Campisi, J. (1995) Proc. Natl. Acad. Sci. USA 92, 9363-9367
18. Robert, L., Fodil-Bourahla, I., Bizbiz, L., and Robert, A. M. (2004) Biomed. Pharmacother. 58, 65-70
19. Gosselin, K., and Abbadie, C. (2003) Exp. Gerontol. 38, 1271-1283
20. Park, W. Y., Park, J. S., Cho, K. A., Kim, D. I., Ko, Y. G., Seo, J. S., and Park, S. C. (2000) J. Biol. Chem. 275, 20847-20852
21. Hailstones, D., Sleer, L. S., Parton, R. G., and Stanley, K. K. (1998) J. Lipid Res. 39, 369-379
22. Nakamura, M., Kondo, H., Shimada, Y., Waheed, A. A., and Ohno-Iwashita, Y. (2003) Exp. Cell Res. 290, 381-390
23. Chung, J. H., Seo, J. Y., Choi, H. R., Lee, M. K., Youn, C. S., Rhie, G. E., Cho, K. H., Kim, K. H., Park, K. C., and Eun, H. C. (2001) J. Invest. Dermatol. 117, 1218-1224
24. Han, J. H., Roh, M. S., Park, C. H., Park, K. C., Cho, K. H., Kim, K. H., Eun, H. C., and Chung, J. H. (2004) Mech. Ageing Dev. 125, 359-366
25. Mouithys-Mickalad, A., Deby-Dupont, G., Dogne, J. M., Leval, X., Kohnen, S., Navet, R., Sluse, F., Hoebeke, M., Pirotte, B., and Lamy, M. (2004) Biochem. Biophys. Res. Commun. 325, 1122-1130
26. Tegeder, I., Pfeilschifter, J., and Geisslinger, G. (2001) FASEB J. 15, 2057-2072
27. Williams, T. M., and Lisanti, M. P. (2004) Genome Biol. 5, 214
28. Cho, K. A., Ryu, S. J., Park, J. S., Jang, I. S., Ahn, J. S., Kim K. T., and Park, S. C. (2003) J. Biol. Chem. 278, 27789-27795
29. Cho, K. A., Ryu, S. J., Oh, Y. S., Park, J. H., Lee, J. W., Kim, H. P., Kim, K. T., Jang, I. S., and Park, S. C. (2004) J. Biol. Chem. 279, 42270-42278
30. Burger, K., Gimpl, G., and Fahrenholz, F. (2000) Cell. Mol. Life. Sci. 57, 1577-1592
31. Dimri, G. P., and Campisi, J. (1994) Exp. Cell Res. 212, 132-140
32. Reiter, R. J., Tan, D., and Burkhardt, S. (2002) Mech. Ageing Dev. 123, 1007-1019
33. Lorenzini, A., Hrelia, S., Bordoni, A., Biagi, P., Frisoni, L., Marinucci, T., and Cristofalo, V. J. (2001) Exp. Gerontol. 36, 65-78
34. Takeda, K., Gosiewska, A., and Peterkofsky, B. (1992) J. Cell. Physiol. 153, 450-459
35. Kang, M. K., Kameta, A., Shin, K. H., Baluda, M. A., Kim, H. R., and Park, N. H. (2003) Exp. Cell Res. 287, 272-281.
36. Hui, A. Y., Dannenberg, A. J., Sung, J. J. Y., Subbaramaiah, K., Du, B., Oling a, P., and Friedman, S. L. (2004) J. Hepatology 41, 251-258

1. (canceled)

2. (canceled)

3. (canceled)

4. (canceled)

5. (canceled)

6. A method for inhibiting cellular senescence, the method comprising treating aged cells with an effective amount of N-[2-(cyclohexyloxyl)-4-nitrophenyl]-methanesulfonamide.

7. A method for regulating the senescence of cells of mammals (except for humans) in need of regulation of cellular senescence, the method comprising administering an effective amount of N-[2-(cyclohexyloxyl)-4-nitrophenyl]-methanesulfonamide to a patient.

8. The method of claim 7, wherein the patient is selected from among patients having Alzheimer's disease, Parkinson's disease, Huntington's disease, stroke, degenerative joint disease, dermal atrophy, elastolysis, sebaceous gland hyperplasia, senile lentigo, graying of hair, hair loss, chronic skin ulcers, osteoporosis, atherosclerosis, calcification, thrombosis, macular degeneration and aneurysms.
